(19) United States
(12) Patent Application Publication
(10) Pub. No.: US 2025/0273316 A1
(43) Pub. Date: Aug. 28, 2025

(54) CONTROLLED INVENTORY REFRIGERATED DISPENSING SYSTEM

(71) Applicant: Minibar Systems, Rockville, MD (US)

(72) Inventors: Mark T. Cosgrove, Edgewater, MD (US); Paul Knox, Carmarthenshire (GB); Alan K. Stratton, Manchester, NH (US); George Bonnoitt, Manchester, NH (US)

(21) Appl. No.: 19/005,152

(22) Filed: Dec. 30, 2024

Related U.S. Application Data
(63) Continuation of application No. 15/629,890, filed on Jun. 22, 2017, now abandoned.
Application No. 15/629,890 is a continuation-in-part of application No. 14/259,621, filed on Apr. 23, 2014, now Pat. No. 9,734,303.
(60) Provisional application No. 61/815,045, filed on Apr. 23, 2013.

(51) Int. Cl. G16H 20/13; A61J 1/16; A61J 7/00; B65D 83/04; F25D 11/00; F25D 25/02; F25D 29/00; G06Q 10/087; G06Q 10/10; G06Q 30/04; G07F 9/00; G07F 9/02; G07F 9/10; G07F 11/18; G07F 17/00; G16H 40/20; G16H 40/63

(52) U.S. Cl. CPC G16H 20/13; A61J 1/165; A61J 7/0076; B65D 83/0409; F25D 11/00; F25D 25/02; F25D 25/025; F25D 29/00; G07F 9/001; G07F 9/002; G07F 9/026; G07F 9/10; G07F 9/105; G07F 11/18; G07F 17/0092; G16H 40/63; A61J 2205/60; F25D 2500/06; F25D 2700/06; F25D 2700/08; G06Q 10/087; G06Q 10/10; G06Q 30/04; G16H 40/20

(57) ABSTRACT

A refrigerated dispensing system is provided including an outer refrigerator cabinet and an interior, removable chassis that mounts a plurality of separately removable product storage and dispensing cartridges. While individual cartridges are removably positionable within slots on one or more shelves inside of the chassis, the entire chassis (with cartridges positioned thereon) may be removed from the outer refrigerator cabinet, as may be desirable in the event of a loss of power to the outer refrigerator cabinet. The shelves are preferably of a modular and reconfigurable design, thus allowing portions of the chassis (e.g., individual shelves or portions thereof) to be modified or replaced with alternative shelving or other storage devices to meet the current refrigerated product storage needs of any particular facility.

### CROSS-REFERENCE TO RELATED APPLICATIONS

This application is a continuation of U.S. patent application Ser. No. 15/629,890, entitled “CONTROLLED INVENTORY REFRIGERATED DISPENSING SYSTEM,” filed on Jun. 22, 2017, which is a continuation-in-part of U.S. patent application Ser. No. 14/259,621, entitled “CONTROLLED INVENTORY REFRIGERATED DISPENSING SYSTEM,” filed on Apr. 23, 2014, which application claims priority under 35 U.S.C. § 119(e) of U.S. Provisional Application Ser. No. 61/815,045, entitled “CONTROLLED INVENTORY REFRIGERATED DISPENSING SYSTEM” filed on Apr. 23, 2013, the specifications of which are incorporated herein by reference in its entirety.

### BACKGROUND OF THE INVENTION

### Field of the Invention

This invention relates generally to refrigerated product storage and dispensing, and more particularly to a refrigeration system for storing products (such as pharmaceutical products) and dispensing such products in a controlled manner that allows automatic tracking of inventory dispensed by and maintained within the refrigerated dispensing unit.

### Background

Pharmaceutical products are distributed in many different ways. In many instances, the pharmaceutical product must be kept at a specific temperature in order to ensure its active ingredients retain their chemical properties. For example, some vaccines and antibiotics must be kept refrigerated to ensure that they maintain their pharmaceutical capabilities. In dispensaries, whether pharmacies or doctor's offices, these drug products are typically kept in traditional refrigerators. There is a minimum level of security and maintaining an inventory of the products can be a tedious and labor intensive endeavor.

Existing refrigerators and other such containers do not provide a doctor or pharmacist with an easy and secure method for dispensing medicines that require refrigeration. Thus, there is a need for a refrigerated container that allows physicians, pharmacists, and other individuals responsible for dispensing medicine to easily dispense the products and maintain an accurate inventory of the products.

Moreover, many systems for dispensing pharmaceutical products are maintained in a secure assembly, such as a locked cabinet, to prevent unauthorized access. However, in the event that such system is refrigerated, and must maintain its contents in a refrigerated condition, upon a loss of power it may become difficult if not impossible to remove product to alternative, refrigerated storage. Likewise, for systems that store many different pharmaceutical products, it may be a significantly time-intensive task to remove each pharmaceutical product from the dispensing system so as to place it in an alternative, refrigerated storage location. Thus, there is a need for a refrigerated container for pharmaceutical or other valuable products that allows quick, alternative access to locked contents during a loss of power, and that allows easy removal of such contents in bulk during such conditions.

Further, varied medical facilities have varying pharmaceutical products stored on hand from time to time, and thus have pharmaceutical product storage needs that may vary over time. Different pharmaceutical products may be provided in packages of varying sizes, such that one dispensing system for such products may not be suitable for all pharmaceutical products stored in any given facility. Thus, there is a need for a pharmaceutical or other valuable product storage and dispensing system that is adaptable so as to be able to securely store and dispense varied products over time.

### SUMMARY OF THE INVENTION

The present invention addresses one or more of the problems described above and other issues associated with traditional storage of refrigerated products that require a controlled inventory. A refrigerated dispensing system is provided including an outer refrigerator cabinet and an interior, removable chassis that mounts a plurality of separately removable product storage and dispensing cartridges. While individual cartridges are removably positionable within slots on one or more shelves inside of the chassis, the entire chassis (with cartridges positioned thereon) may be removed from the outer refrigerator cabinet, as may be desirable in the event of a loss of power to the outer refrigerator cabinet. The shelves are preferably of a modular and reconfigurable design, thus allowing portions of the chassis (e.g., individual shelves or portions thereof) to be modified or replaced with alternative shelving or other storage devices to meet the current refrigerated product storage needs of any particular facility.

With regard to a particular embodiment, a refrigerated storage and dispensing unit is provided, comprising a refrigerator cabinet, a chassis positioned on an interior of the refrigerator cabinet having walls defining an open cavity on an interior of the chassis, a shelf affixed to the walls of the chassis within the open cavity, and a plurality of product dispensing cartridges removably positioned on the shelf, each product dispensing cartridge further comprising a vertical housing holding a plurality of product units and a dispensing drawer configured for horizontal movement out of the housing, wherein each product dispensing cartridge is configured to sequentially dispense a single one of the product units upon each horizontal extension of the dispensing drawer from a fully closed position to a fully open position.

### BRIEF DESCRIPTION OF THE DRAWINGS

The present invention and various aspects, features, and advantages provided by it are described in detail below with reference to exemplary and non-limiting embodiments and with reference to the drawings, which constitute part of this specification and provide graphic depictions of certain exemplary embodiments. The following is a brief description of the drawings:

FIG. 1 is a front perspective view of a refrigerated storage and dispensing unit in accordance with certain aspects of an embodiment of the invention.

FIG. 2 is a block diagram of the control and chilled compartment.

FIG. 3A is a front perspective view of the refrigerated storage and dispensing unit of FIG. 1 with its front door open to provide access to the interior of the unit.

FIG. 3B is a front perspective view of the refrigerated storage and dispensing unit of FIG. 3A with dispensing cartridges positioned on a bottom shelf.

FIG. 4 is a front perspective view of an interior chassis for use with the refrigerated storage and dispensing unit of FIG. 1.

FIG. 5 is an exploded view of the interior chassis of FIG. 4.

FIG. 6 is a close-up view of a chassis release system for use with the interior chassis of FIG. 4.

FIG. 7 is a front view of the chassis of FIG. 4.

FIG. 8 is a perspective view of a combined storage shelf and storage drawer for use with the chassis of FIG. 4.

FIG. 9 is a close-up view of the combined storage shelf and storage drawer of FIG. 8.

FIG. 10 is a front view of the combined storage shelf and storage drawer of FIG. 8.

FIG. 11 is a perspective view of the combined storage shelf and storage drawer of FIG. 4 with the printed circuit board and bottom plate removed for clarity.

FIG. 12 is a perspective view of the combined storage shelf and storage drawer of FIG. 11 with only the printed circuit board removed for clarity.

FIG. 13 is a side perspective view of the combined storage shelf and storage drawer of FIG. 11.

FIG. 14 is a top perspective view of the combined storage shelf and storage drawer of FIG. 11.

FIG. 15 is a rear perspective view of the combined storage shelf and storage drawer of FIG. 11.

FIG. 16 is a close-up rear perspective view of the combined storage shelf and storage drawer of FIG. 11.

FIG. 17 is a front perspective view of a dispensing cartridge for use with the storage and dispensing unit of FIG. 1.

FIG. 18 is a rear perspective view of the dispensing cartridge of FIG. 17.

FIG. 19 is a cross sectional view of an RFID open-front box for use with the storage and dispensing unit of FIG. 1.

FIG. 20 is a rear view of the RFID open-front box of FIG. 19.

FIG. 21 is a block diagram of a freezer compartment for use with the storage and dispensing unit of FIG. 1.

FIG. 22 is a schematic diagram of a system in accordance with certain aspects of embodiment of the invention.

FIG. 23 is a schematic diagram of a network for implementing the system of FIG. 22.

FIG. 24 is a flow diagram of a process for dispensing products in accordance with further aspects of an embodiment of the invention.

### DETAILED DESCRIPTION

The invention summarized above may be better understood by referring to the following description, which should be read in conjunction with the accompanying claims and drawings in which like reference numbers are used for like parts. The following description is of a particular embodiment of the invention, set out to enable one to practice an implementation of the invention, and is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

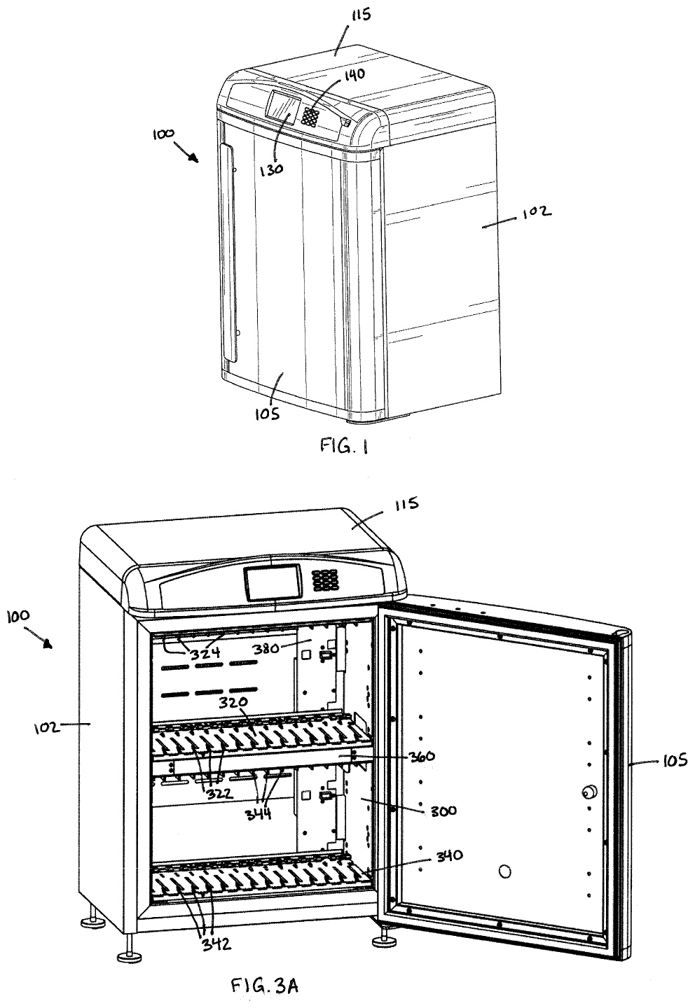

First, with reference to FIG. 1, an exemplary refrigerated storage and dispensing unit 100 according to certain aspects of an embodiment of the invention consists of a refrigerator cabinet 102 having a door 105 and a control compartment 115, which control compartment 115 may include a communication interface configured to communicate with a remote server. Refrigerator cabinet 102 may include a chiller (most preferably ammonia absorption, but in certain embodiments a compressor will be used).

A system utilizing such refrigerated storage and dispensing unit may be operated by a person removing a product from an interior of refrigerator cabinet 102, and control compartment 115 recording the removal of product as a transaction in its local memory on the control compartment 115. Periodically, the control compartment 115 may use the communication interface to send transaction data along with events (e.g., historical temperature data, power outages, events causing alarms, etc.) to a remote server. The remote server may then communicate with outside entities for inventory control, maintenance, alarms, billing, and any other essential tasks. A refill technician or specialist may communicate with the refrigerated storage and dispensing unit 100 using a display 130 and keypad 140 on the control compartment 115, or optionally via a web-enabled device, BLUETOOTH device, or other remote device to manage the functions and refilling of the refrigerated storage and dispensing unit 100.

Preferably, external door 105 is provided an electric or mechanical lock, and is used to control access to the products stored in the refrigerated storage and dispensing unit 100. If an electric lock is provided, such electric lock is preferably controlled by a computer processor in the control compartment 115 after a valid access code is entered or, as described more fully below, when specific events occur that require that the door be locked or unlocked.

Control compartment 115 provides a user interface that a pharmacist, doctor, or other user may engage to manage the refrigerated storage and dispensing unit 100. In accordance with certain aspects of an embodiment of the invention and as noted above, control compartment 115 preferably includes a display 130 and a user input device 140. Display 130 may consist of a LCD, and the user input device may be a key pad. Optionally, the user input device 140 and the display 130 may be combined into a touchscreen as will be recognized by a person of ordinary skill in the art. The control compartment 115 may also optionally include one or more of the following elements: an alarm sounder, a camera for a bar code reader, a USB communications interface, an external communications interface (Ethernet, PLC, POTS, Cellular, Satellite, WiFi, etc.), and an internal communications interface (I2C and GPIO) for communication among the internal components of the refrigerated storage and dispensing unit (as discussed in greater detail below), all controlled by a computer processor.

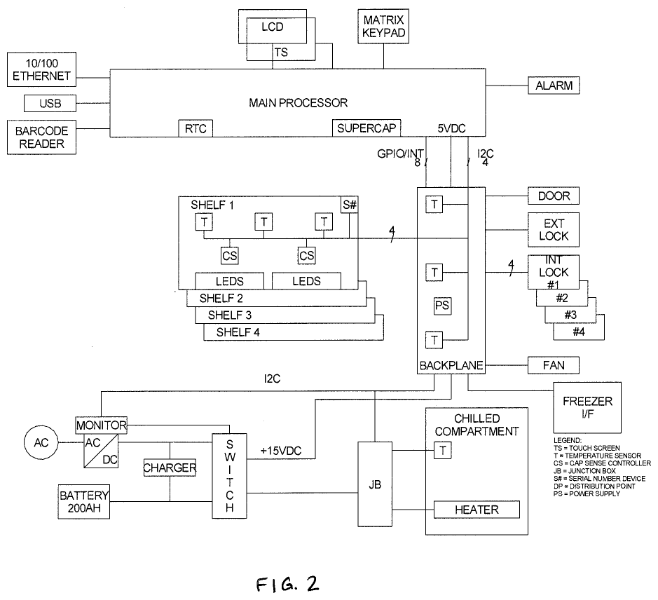

Control compartment 115 preferably includes the computer processor, memory, RTC, battery backup, and necessary interfaces to communicate with all hardware. The computer processor may use a Linux OS with an Android OS and application running on it, although alternative operating systems may be employed. FIG. 2 provides a schematic diagram of the electrical connections of an exemplary control compartment 115.

Preferably, all control and communications are managed by control compartment 115. Users communicate with the vending refrigerator 100 via the user interface, e.g., keypad 140 and LCD 130, touchscreen, or web or BLUETOOTH enabled device such as a smart phone, tablet, laptop, etc. Communications to a server and external users are accomplished via the external communications interface. The user interface may be managed through a mobile device application to control the screens, touchscreen and keypad. The same mobile application or a remote website can be used in a mobile device and, thus, the user does not have to be co-located with the refrigerated storage and dispensing unit 100 in order to affect such control. The mobile application or remote website enables any wireless communication device to interact with the refrigerated storage and dispensing unit 100 or the system that includes the refrigerated storage and dispensing unit 100.

An alarm sounder may be used to signify to local users that a critical event (e.g., no power, temperature out of range, etc.) has occurred and that a user is needed to attend to the refrigerated storage and dispensing unit 100. The user can mute the alarm, e.g., via LCD 130 and/or keypad 140. A USB interface preferably provides a maintenance port for diagnostics and emergency download of data. It is contemplated that the interface may be any port, USB or otherwise, that allows physical electronic access to the internal components of the refrigerated storage and dispensing unit 100, e.g., Ethernet port, HDMI, etc. Such interface may also be used to setup the refrigerated storage and dispensing unit 100 and for manufacturing assistance. A barcode reader, which consists of a camera, camera interface, and an application to manage the reader, e.g., a mobile device or wireless device, can be used for reading data from the products to be stored in the refrigerated storage and dispensing unit 100. Typical data may include the kind of product, expiration date and lot code.

The main power supply is preferably monitored to determine the type of current being supplied, e.g., AC or DC, the current voltage, and the current battery status. The interface is also used to control the switch between AC and a battery source for testing the battery and during loss of AC power. The switch is also used to shed the load of the chiller if the battery supply runs low. A backup power supply on the processor board consisting of a battery or supercapacitor is used in the event that the main power source is not present. In the event that the main power source is not present, software executable by control compartment 115 will load the volatile data into non-volatile memory (Flash, MRAM, FRAM, EEPROM, etc.) using this backup power supply.

Internal communications among the components of the refrigerated storage and dispensing unit 100 (discussed in detail below) may be managed via I2C of GPIO. The I2C is preferably used for the main communications for control and status. The GPIO is preferably used for real time interrupts and programming.

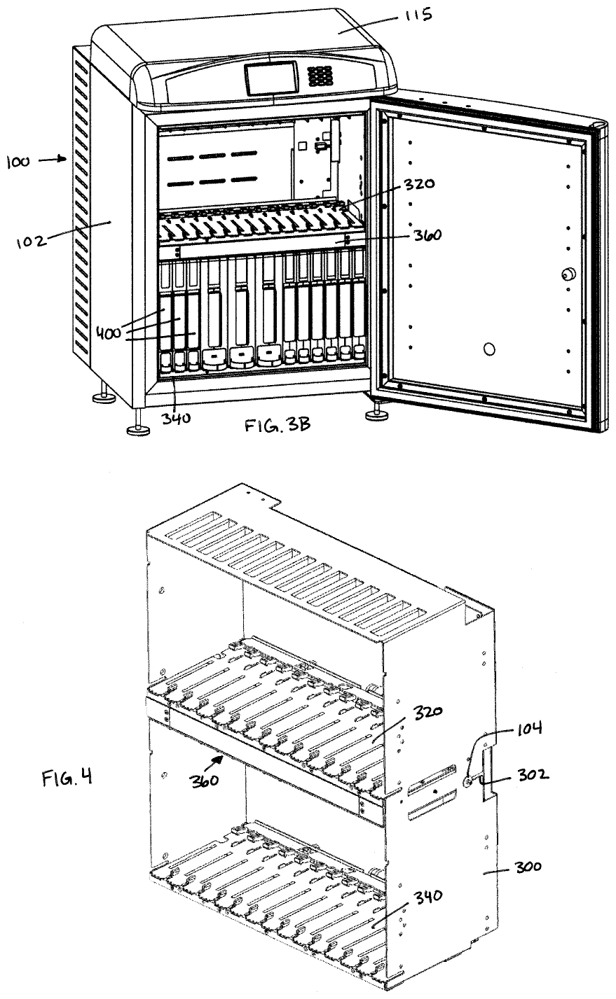

FIGS. 3a and 3b show door 105 of refrigerator cabinet 102 open to provide access to product stored within the refrigerated storage and dispensing unit 100. As shown in FIG. 3a and in accordance with certain aspects of an embodiment of the invention, the refrigerated storage and dispensing unit may include an interior chassis 300, a top shelf 320, a bottom shelf 340, and an intermediate drawer assembly 360, all of which are discussed in greater detail below. Top shelf 320 and bottom shelf 340 define parallel slots configured to receive individual product dispensing cartridges 400 (FIG. 3b), with such product dispensing cartridges arranged parallel to one another along their respective shelf. More particularly, each such product dispensing cartridge 400 holds a plurality of product containers, such as vials of injectable pharmaceuticals, syringe packages, or other such packages as may be suitable to a particular facility or configuration. Moreover, each such product dispensing cartridge 400 includes a dispensing drawer at the base of each cartridge, with each such dispensing cartridge 400 being configured to dispense only a single product container at a time from its respective dispensing drawer. The product dispensing cartridges 400 are insertable into slots on shelf 320 and shelf 340. Such slots for top shelf 320 are defined by guide tabs 322 extending upward from shelf 320 and dividers 324 extending downward from an upper divider wall defined by the top wall of chassis 300, and slots for bottom shelf 340 are defined by guide tabs 342 extending upward from shelf 340 and dividers 344 extending downward from a lower divider wall defined by the bottom wall of intermediate drawer assembly 360. Further and as discussed in detail below, product dispensing cartridges 400 may be locked in place on shelf 320 and on shelf 340 with movable latches that engage the rear of each product dispensing cartridge 400.

Each of top shelf 320 and bottom shelf 340 preferably includes sensors that engage with product dispensing cartridges 400 (shown in FIG. 3b) to detect dispensing of product from such dispensers 400. In order to collect such data from top shelf 320 and bottom shelf 340, a backplane 380 is located at the back of chassis 300, and dispensing shelves 320 and 340 electronically connect to backplane 380 to transfer data collected by sensors on dispensing shelves 320 and 340 to control compartment 115. Backplane 380 buffers the communication signals between the top shelf 320 and bottom shelf 340 and the processor of control compartment 115. Temperature sensors on backplane 380 may provide temperature data to the processor of control compartment 115. Controls for a fan and door locks are preferably located on backplane 380 and are in communication with the processor of control compartment 115. The door sensor logic is preferably stored on backplane 380 for the processor of control compartment 115 to access. Control logic on backplane 380 may determine which shelf, device, or freezer interface the processor platform communicates with. Power from the main power supply is distributed on backplane 380 for shelves 320 and 340 and logic. Power is preferably stepped down on backplane 380 for the processor in control compartment 115.

As mentioned above, top shelf 320 and bottom shelf 340 receive product dispensing cartridges 400, with such product dispensing cartridge sitting on top of a shelf 320 or 340, with contacts on the movable dispensing drawer of each product dispensing cartridge positioned and otherwise configured to activate the sensors on the shelf (discussed in greater detail below). In accordance with certain aspects of an embodiment of the invention, each of shelf 320 and 340 has several sensor pads under each product dispensing cartridge 400. More particularly, and by way of non-limiting example, each of shelf 320 and 340 may have at least three sensor pads facing upwards in a configuration that allows product and drawer sensing as described in more detail below. The sensor pads are connected to a processor on the respective shelf 320 or 340, or directly to the processor of control compartment 115 through backplane 380. The sensors can be, by way of non-limiting example, capacitive, infrared, or mechanical. A sensing application running on the processor detects the presence or absence of product on that particular sensor, and may also detect the mechanical position of different parts of the product dispensing cartridge. This data is sent to the processor of control compartment 115 to determine if the product has been removed or if there is a malfunction. Data from a digital temperature sensor is also sent to the processor of control compartment 115 to be used in controlling a heater/cooler, for historical data for product aging, and for alarms.

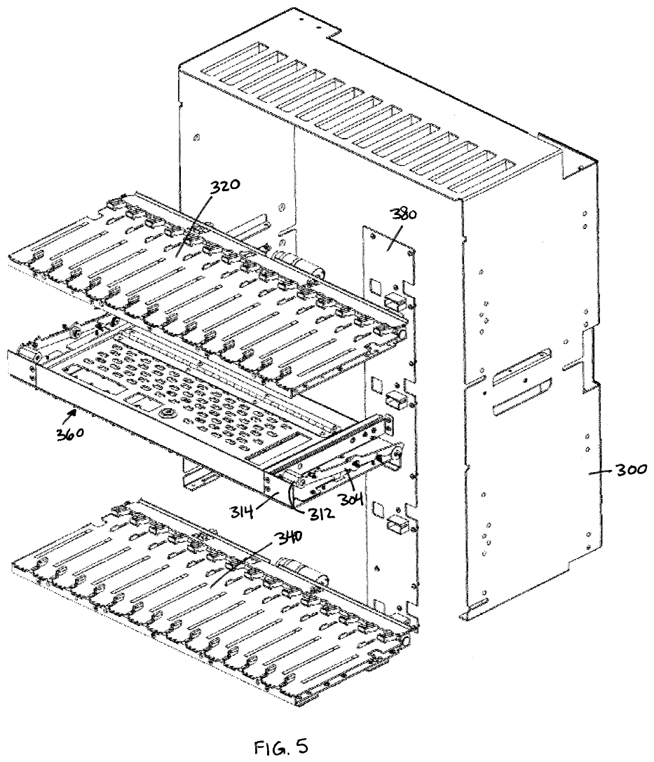

FIG. 4 provides a perspective view, and FIG. 5 an exploded perspective view, of chassis 300 removed from refrigerator cabinet 102 (refrigerator cabinet 102 not shown for clarity). Circumstances may arise that create a desirability or need to remove the entire contents of the refrigerated storage and dispensing system 100, such as in the event of a long-term power loss which would require moving all contents to an alternative refrigerated storage. To provide for such capability, the entire chassis 300 is removable from the interior of refrigerator cabinet 102. A refrigerator cabinet mounting nut 104 is fixedly mounted to at least one, and preferably both interior sidewalls of refrigerator cabinet 102. Chassis 300 has a horizontal mounting nut slot 302 in preferably both sidewalls of chassis 300, which mounting nut slot 302 slidably receives refrigerator cabinet mounting nut 104. As shown in FIG. 5 and FIG. 6 (in which chassis 300 is shown in phantom), mounting nut 104 serves as an anchor to releasably hold chassis 300 in refrigerator cabinet 102. A chassis release bar 304 is pivotably mounted to intermediate drawer assembly 360 at pivot screw mount 306. As best viewed in the close-up side perspective view of intermediate drawer assembly 360 and top shelf 320 of FIG. 9, chassis release bar 304 has a notch 308 at a back end of chassis release bar 304, which notch 308 receives refrigerator cabinet mounting nut 104. While chassis release bar 304 is pivotable about pivot screw mount 306, a spring 310 biases chassis release bar 304 into a position that engages notch 308 with mounting nut 104, in turn locking chassis 300 in refrigerator cabinet 102. A handle 312 is provided at the front end of chassis release bar 304, which handle 312 may be manually engaged and pushed downward to pivot chassis release bar 304 about pivot screw mount 306, disengaging notch 308 from mounting nut 104, and thus allowing the entire chassis 300 (along with top shelf 320, bottom shelf 340, and drawer assembly 360) to be horizontally pulled outward from refrigerator cabinet 102. Preferably, a release bar cover plate 314 is removably mounted by, for example, security screws to the front face of intermediate drawer assembly 360, allowing an authorized person to access handle 312 to disengage chassis release bar 304 from mounting nut 104 and remove the entire chassis and its contents from the refrigerator cabinet 102.

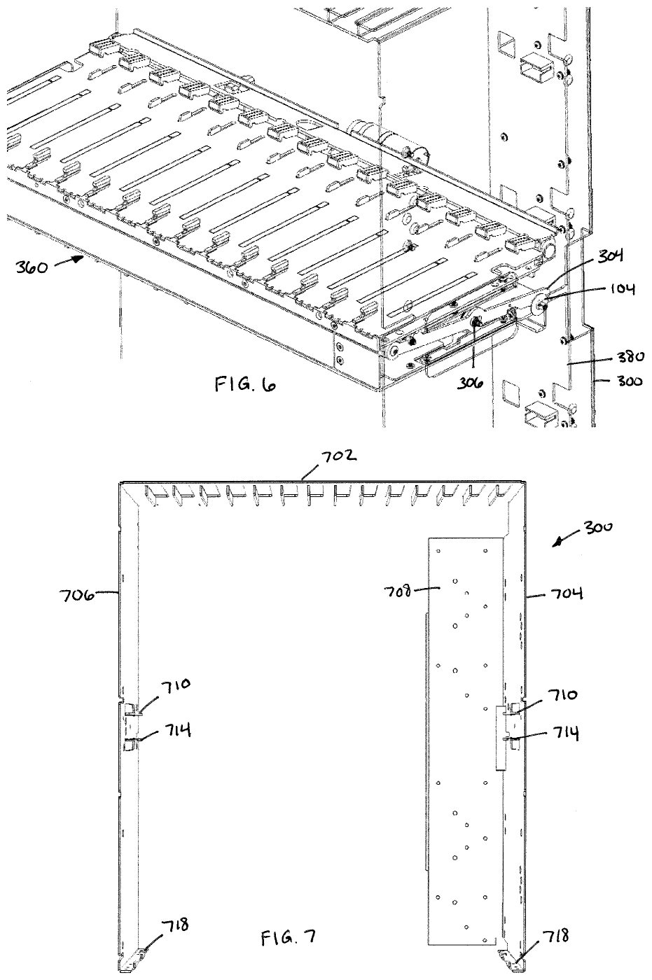

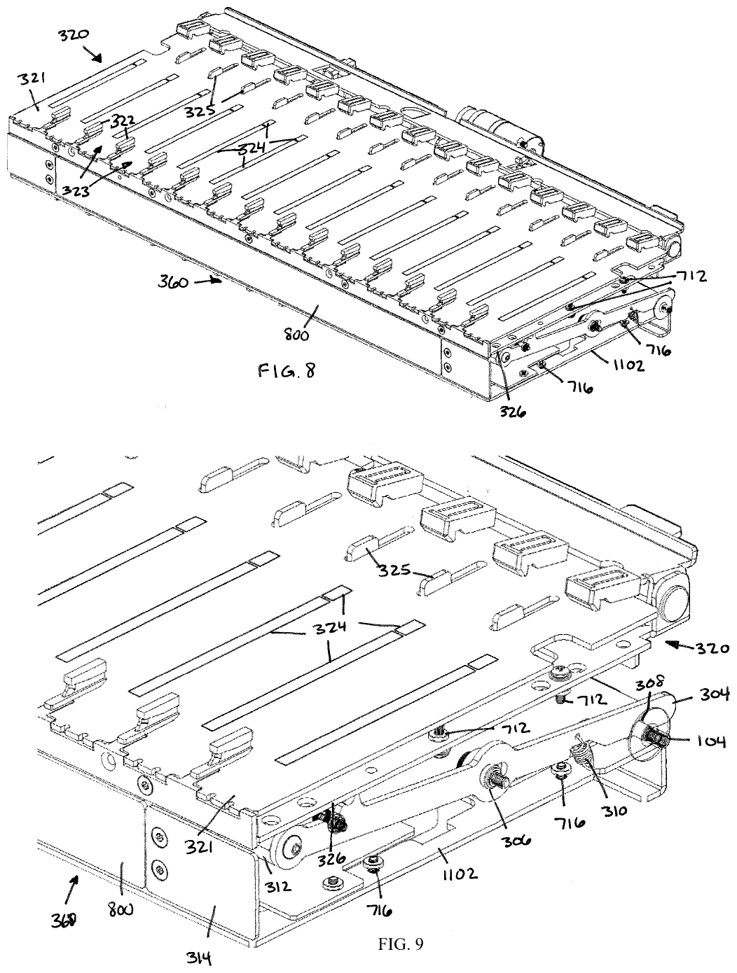

As mentioned above, top shelf 320, bottom shelf 340, and intermediate drawer assembly 360 are mounted to chassis 300. FIG. 7 is a front view of chassis 300 with top shelf 320, bottom shelf 340, and intermediate drawer assembly 360 removed for clarity. Chassis 300 includes a top wall 702, a right side wall 704, and a left side wall 706. Chassis 300 also provides a mounting wall 708 for backplane 380. Right side wall 704 and left side wall 706 include upper support flanges 710 extending into the interior of chassis 300 from each respective sidewall. Upper support flanges 710 receive and support the bottom wall of top shelf 320, such as by way of threaded connectors 712 (FIGS. 8 and 9). Right side wall 704 and left side wall 706 also include lower support flanges 714 extending into the interior of chassis 300 from each respective sidewall. Lower support flanges 714 receive and support the bottom wall of intermediate drawer assembly 360, such as by way of threaded connectors 716. Still further, right side wall 704 and left side wall 706 include bottom mounting brackets 718 extending into the interior of chassis 300 from each respective sidewall. As with upper support flanges 710, bottom mounting brackets 718 receive and support the bottom wall of bottom shelf 342, such as by way of threaded connectors.

Such support configuration allows replacement of any of top shelf 320, bottom shelf 340, or intermediate drawer assembly 360 with minimal effort, as may be desirable to meet changing refrigerated product storage needs. By way of non-limiting example, a given facility may wish to dedicate only bottom shelf 340 to receiving cartridges 400, and may wish to provide alternative storage space in the top portion of chassis 300. In this case, top shelf 320 may be removed from chassis 300 by removing threaded connectors 712, and such alternative storage assembly as may be selected by persons skilled in the art may be affixed in its place. In certain configurations, an open-front storage box equipped with a radio frequency identification (“RFID”) antenna may be put in the place of top shelf 320 and attached to upper support flanges 710, or positioned directly on top shelf 320 or a portion thereof (such as to bottom plate 326 of top shelf 320, discussed below), which may receive and store RFID tagged product containers, as discussed in greater detail below.

With continued reference to FIGS. 7 and 8, top shelf 320 includes a flat panel 321 that serves as the support for product dispensing cartridges 400. In certain configurations, flat panel 321 comprises a printed circuit board. Guide tabs 322 are positioned adjacent a front edge of flat panel 321 and serve to properly position product dispensing cartridges 400 as they are being placed on top shelf 320. To further assist in properly positioning product dispensing cartridges, rear guide posts 325 are likewise provided and positioned in alignment with guide tabs 322, thus defining individual slots 323 configured to receive a product dispensing cartridge 400. Sensors 324 as discussed above are positioned in the top face of flat panel 321 so as to align with a bottom, horizontally moveable dispensing drawer on each cartridge 400, thus allowing sensors on the bottom of such drawers to activate sensors 324 on flat panel 321, in turn allowing the processor of control compartment 115 to record dispensing of product from a particular product dispensing cartridge 400. Top shelf 320 also includes bottom plate 326, which both mounts top shelf 320 to upper support flanges 710, and positions flat panel 321 a vertical distance above bottom plate 326.

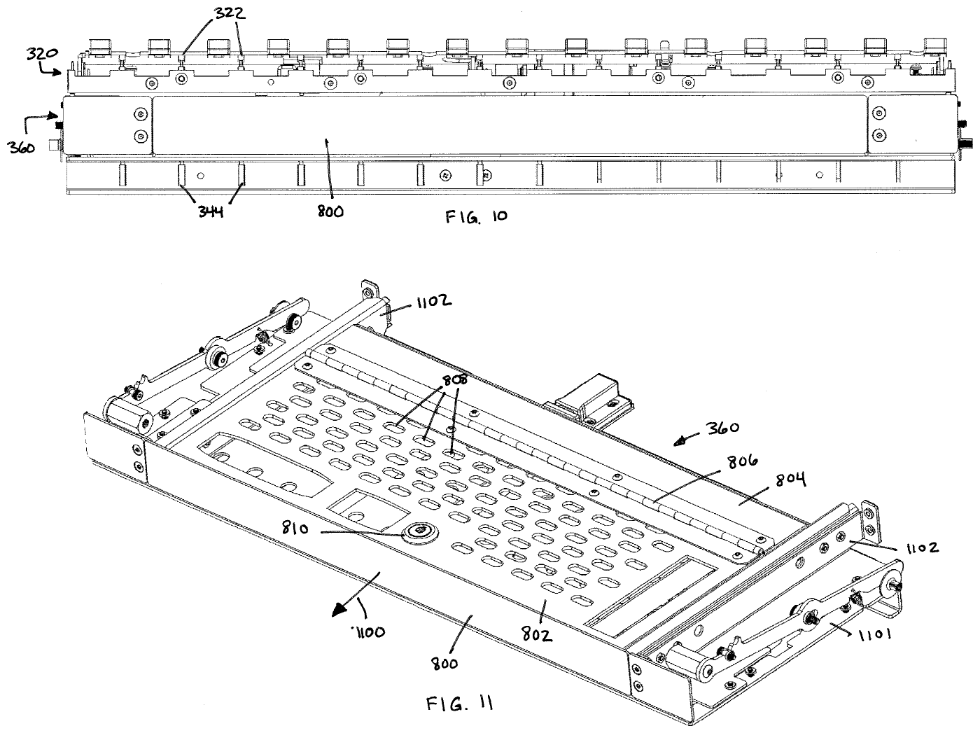

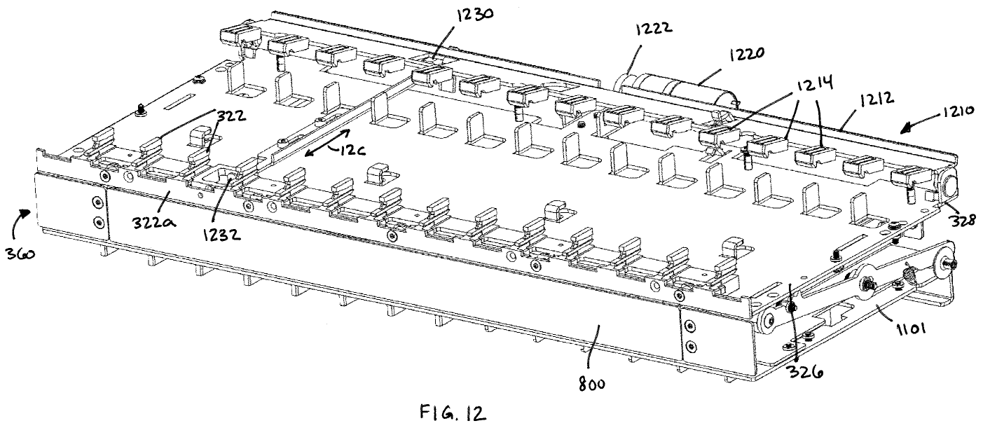

As shown in FIGS. 8-11, while top shelf 320 is positioned immediately above intermediate drawer assembly 360, as discussed above it is independently mounted to chassis 300, allowing drawer assembly 360 to slide horizontally outward (in the direction of arrow 1100 in FIG. 11) from chassis 300 for placement and retrieval of product. As best viewed in FIG. 11 (in which flat panel 321 has been removed for clarity), drawer front 800 may be pulled outward in the direction of arrow 1100 from intermediate drawer assembly 360, such as by way of standard drawer slide rails 1102. Slide rails 1102 are attached to both sides of drawer assembly 360, allowing the drawer to be pulled outward from chassis 300 over drawer assembly support plate 1101 without effecting top shelf 320 or bottom shelf 342, or the dispensing cartridges 400 stored on them. Drawer assembly 360 includes a hinged lid 802, hinged to drawer body 804 via a hinge member 806. Hinged lid 802 is preferably perforated with a plurality of openings 808 allowing cold air to pass through drawer assembly 360 so as to keep contents of drawer assembly 360 cold with the rest of the product stored in refrigerated storage and dispensing unit 100. Hinged lid 802 may be provided a separate lock 810 to provide restricted access to the contents of drawer assembly 360. Drawer assembly 360 may be useful for storing product that was improperly or inadvertently withdrawn from dispensing cartridges, product that has already been dispensed but only partially used, or product in oddly shaped containers that do not fit into dispensing cartridges 400.

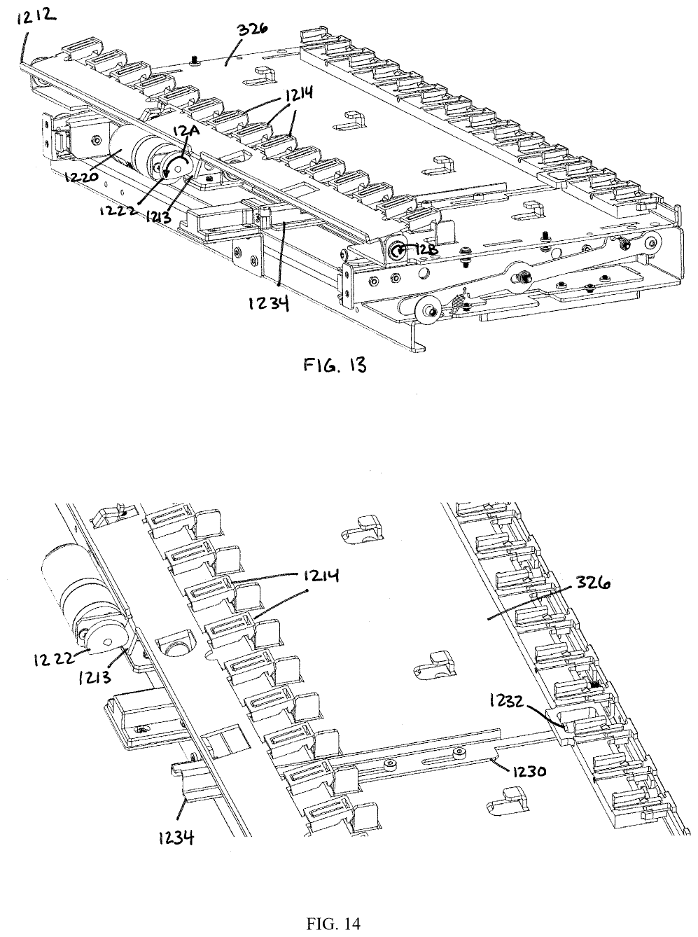

FIG. 12 shows drawer assembly 360 with bottom plate 326 of top shelf 320 in place above drawer assembly 360. Likewise, FIGS. 13 through 16 show drawer assembly with bottom plate 326 removed. Each of FIGS. 12 through 16 show cartridge release mechanism 1210 pivotably mounted to bottom plate 326. More particularly, and with continuing reference to FIGS. 12 through 16, cartridge release mechanism 1210 includes mounting arm 1212 pivotably mounted at pivot tab 328 to bottom plate 326. A plurality of hooks 1214 are mounted to the front face of mounting arm 1212, with one hook 1214 aligned with each slot 323. Hooks 1214 are configured to engage a ridge on the back of each dispensing cartridge 400. Thus, when cartridge release mechanism is in its downward position, hooks 1214 engage the back of dispensing cartridges 400 to prevent their removal from refrigerated storage and dispensing unit 100. When cartridge release mechanism is pivoted to its upward position, hooks 1214 disengage from their respective dispensing cartridges 400, allowing the dispensing cartridges to be removed from their respective shelf 320 and 340. Cartridge release mechanism 1210 is preferably spring biased to its downward position, so as to prevent removal of dispensing cartridges 400 absent an affirmative and authorized action to engage/lift cartridge release mechanism 1210.

In order to cause cartridge release mechanism 1210 to lift hooks 1214, an actuator controlled by the processor, and more particularly a drive motor 1220, is positioned at the back of bottom plate 326. Drive motor 1220 drives a cam wheel 1222, which cam wheel 1222 engages actuator 1213 on mounting arm 1212. Specifically, as cam wheel 1222 is rotated by drive motor 1220 in the direction of arrow 12A (FIG. 13), mounting arm 1212 is rotated in the direction of arrow 12B (FIG. 13) to lower hooks 1214 into their downward, locking position. Likewise, as cam wheel 1222 is rotated in the direction opposite of arrow 12A, mounting arm 1212 is rotated in the opposite direction of arrow 12B to raise hooks 1214 into their upward, unlocked position. Drive motor 1220 is preferably operated by control compartment 115 in response to receive instruction from an authorized use to either lock or unlock dispensing cartridges 400 from their respective shelves.

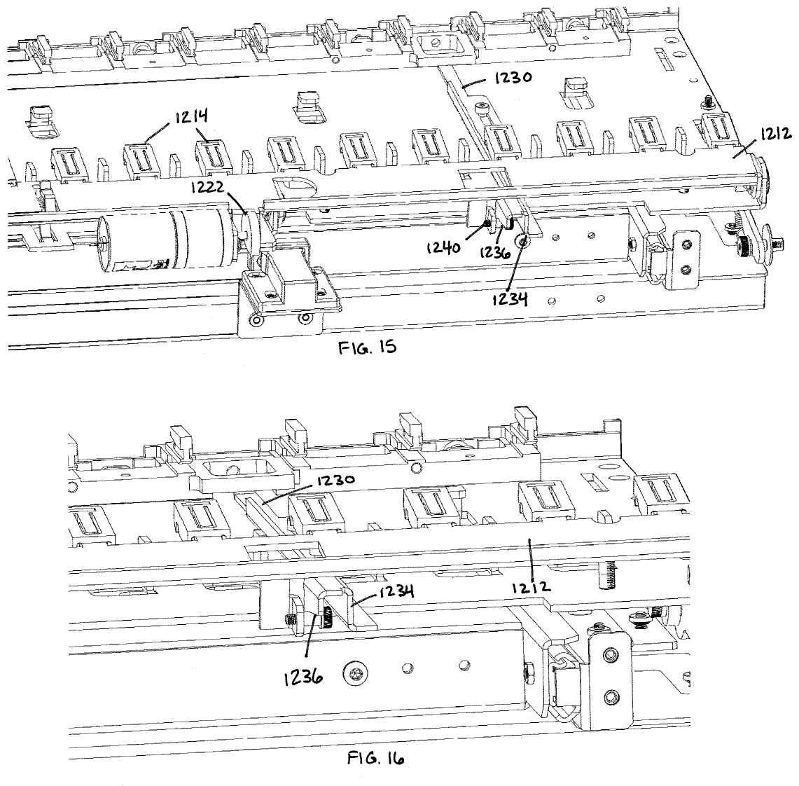

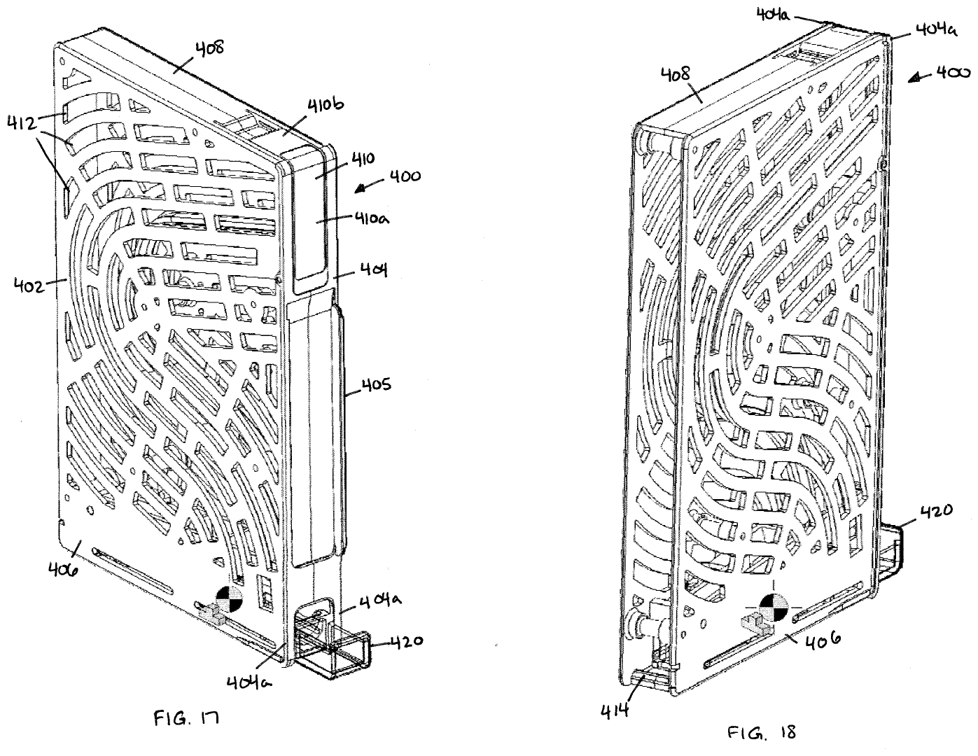

In the event of a power loss, and in the case that during such power loss it is necessary or desirable to remove cartridges 400, a manual release bar 1230 is also provided. Manual release bar 1230 is slidably mounted to the top of bottom plate 326, and is mounted for movement in the direction of arrow 12C (FIG. 12). Release bar 1230 has a pull tab 1232 positioned at the front end of release bar 1230. Preferably, pull tab 1232 is hidden behind the front wall 322a of cartridge guide tabs 322, such that front wall 322a serves as a security panel to prevent unauthorized access to pull tab 1232. Moreover, front wall 322a is preferably mounted to bottom plate 326 with a plurality of security screws, such that one may only access pull tab 1232 with the appropriate tool, and with knowledge that the hidden pull tab 1232 is positioned behind front wall 322a of cartridge guide tabs 322. The back end of release bar 1230 includes a lifting head 1234 that engages the underside of mounting arm 1212. When cam wheel 1222 is rotated so as to allow position hooks 1214 to be pulled to their downward, locking position, release bar 1230 may be pushed to its rearmost position (i.e., the position shown in FIGS. 12 through 16), causing lifting head 1234 to push against mounting arm 1212 and lift mounting arm 1212 and hooks 1214 to their upward, unlocked position, in turn allowing removal of dispensing cartridges 400. As best seen in FIGS. 15 and 16, lifting head 1234 includes a bottom angled edge 1236 that engages a set screw 1240, such that as lifting head 1234 is pushed rearward, bottom angled edge 1236 contacting set screw 1240 causes lifting head 1234 to slightly lift mounting arm 1212, pivoting mounting arm 1212 upward and causing hooks 1214 to raise to their upward, unlocked position.

While FIGS. 12 through 16 show mounting arm 1212 and hooks 1214 and related mechanisms on top shelf 320, an identical assembly is likewise preferably provided for bottom shelf 340 (without intermediate drawer assembly 360).

FIG. 17 shows an exemplary dispensing cartridge 400 for use with the refrigerated storage and dispensing unit 100. Each dispensing cartridge 400 comprises a generally vertical housing 402 and a dispensing drawer 420 at the bottom of housing 402. Dispensing drawer 420 is configured for horizontal sliding out from and back into housing 402. Moreover, as discussed in copending U.S. patent application Ser. No. ______, the specifications of which are hereby incorporated by reference, each dispensing cartridge 400 includes a dispensing mechanism which allows only a single product unit stored in dispensing cartridge 400 (e.g., a single vial of pharmaceutical product, a single box containing a syringe, etc.) to be dispensed with each pull of dispensing drawer 420, blocks such product unit from being reinserted into dispensing cartridge 400 after dispensing drawer 420 has been pulled out of housing 402, prevents closure of the dispensing drawer until removal of the single product unit from the dispensing drawer 420, and prevents reloading of the next such product unit into the dispensing drawer 420 until dispensing drawer 420 is pushed fully back into housing 402. Each dispensing cartridge 400 is configured to deliver the next product unit by gravity feed into the dispensing drawer 420 after the dispensing drawer 420 has been fully closed following a dispensing operation (i.e., full opening of the dispensing drawer 420 from cartridge 400 and removing the dispensed product unit).

Each dispensing cartridge 400 includes a front face 404, two side walls 406, and a top wall 408. When positioned on one of top shelf 320 and bottom shelf 340, front face 410 of each dispensing cartridge 400 faces the front of refrigerator cabinet 102. A product reload door 410 is accessible from front face 404, which may be opened by a user to reload additional product. However, product reload door 410 has both a vertical portion 410a and a horizontal portion 410b, wherein access to horizontal portion 410b is blocked when dispensing cartridge 400 is fully inserted into refrigerated storage and dispensing unit 100. Thus, a user may only open dispensing cartridge 400, and thus access product stored therein, after the authorized removal of the cartridge 400 from refrigerated storage and dispensing unit 100.

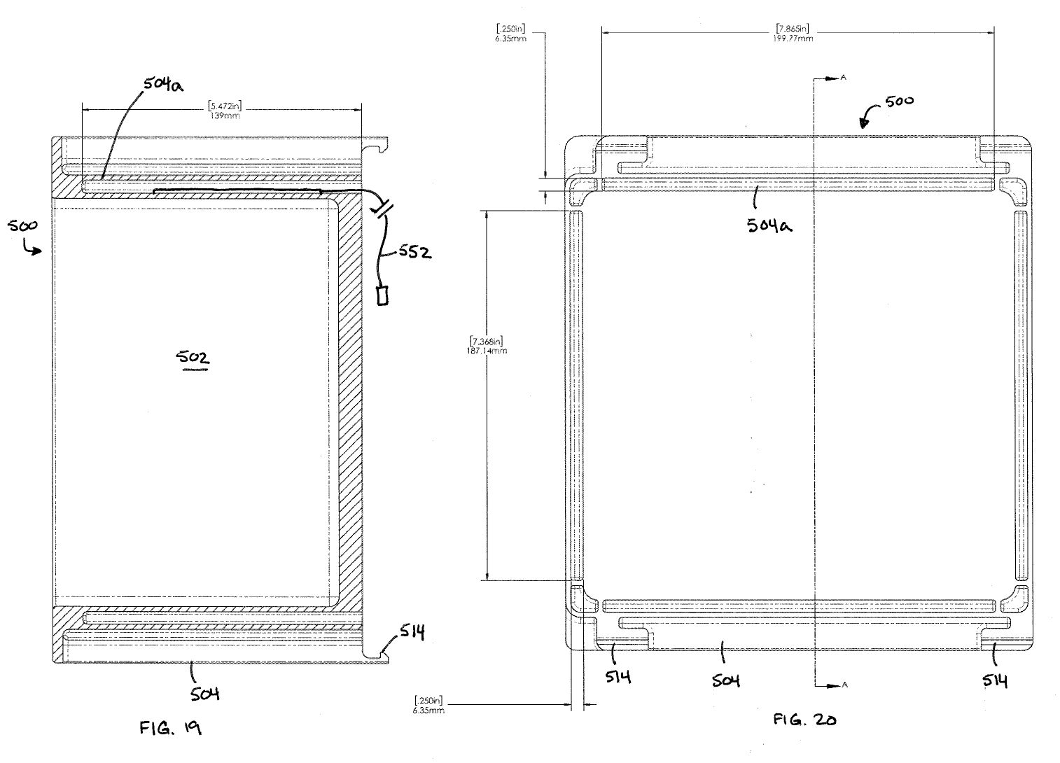

Side walls 406 of dispensing cartridge 400 include a plurality of openings 412. Openings 412, along with the open back (best seen in FIG. 18) of each dispensing cartridge 400, provide airflow around product stored within dispensing cartridge 400 to ensure that all such product remains uniformly refrigerated. Further, front face 404 has a wider width than top wall 408, thus creating a space between adjacent dispensing cartridges 400 when they are positioned on top shelf 320 and bottom shelf 340, further providing for refrigerated airflow in the spaces between adjacent dispensing cartridges 400. The outer edges 404a of front face 404 of adjacent dispensing cartridges 400 abut one another so as to form a relatively uniform front wall with minimal air gap between them, thus keeping as much of the refrigerated air behind the front faces 404 of the dispensing cartridges 400 when the refrigerator cabinet 102 is open. A pull tab 405 may be provided extending vertically along the front face 404 of each dispensing cartridge 400 so as to allow for easy removal of a single dispensing cartridge 400 from top shelf 320 or bottom shelf 340 when desired (and unlocked), such as for refilling purposes.

As best viewed in FIG. 18, horizontal ridge 414 extends across the bottom, rear side of dispensing cartridge 400. Ridge 414 provides an engagement surface against which hooks 1214 of cartridge release mechanism 1210 acts to lock each dispensing cartridge 400 in place on its respective top shelf 320 or bottom shelf 340. More particularly, when hooks 1214 are pivoted to their downward, locking positions, the forward end of a hook 1214 will engage the ridge 414 of an aligned dispensing cartridge 400, thus locking the dispensing cartridge 400 on its respective shelf. Likewise, when hooks 1214 are pivoted to their upward, unlocked position, the forward end of such hook 1214 disengages from the ridge 414 of the aligned dispensing cartridge 400, thus unlocking the dispensing cartridge 400 from its respective shelf and allowing its individual removal from refrigerated storage and dispensing unit 100.

In a particular configuration, dispensing cartridges 400 may include a product sensor that detects the presence of product within dispensing cartridge 400. More particularly, when the dispensing drawer 420 is closed and there is product in the dispensing cartridge 400, the next product to be dispensed may push the front end of a sensing lever downward so as to cause the rear end of such sensing lever to rise, in turn breaking a connection between the product sensor and the sensor on the respective shelf 320 or 340 on which the dispensing cartridge 400 is positioned, thus indicating that there is product in such dispensing cartridge 400 in position for dispensing. When the dispensing drawer 420 is open and there is no additional product pressing the front end of such sensing lever, the rear end of the sensing lever is lowered and connects the product sensor with the sensor on the respective dispensing shelf 320 or 340, indicating that there is currently no product in line to be dispensed. When the dispensing drawer 420 is closed, the next product in line in the dispensing cartridge 400 moves into dispensing drawer 420, pushing down the front end of the sensing lever and causing the rear end of the product sensor to rise and lose its connection with the sensor on the respective dispensing shelf 320 or 340. It is contemplated that other product sensor configurations could be readily implemented by a person of ordinary skill in the art.

In a particular configuration, dispensing cartridges 400 may also include a drawer position sensor located on the bottom of the dispensing drawer 420. The drawer position sensor may connect with a closed position sensor on the respective top shelf 320 and bottom shelf 340 when the dispensing drawer 420 is closed, indicating to the control compartment 115 that the dispensing drawer 420 is closed. The drawer position sensor may also connect with an open position sensor on the respective top shelf 320 and bottom shelf 340 when the dispensing drawer 420 is fully open, indicating to the control compartment 115 that the dispensing drawer 420 is fully open.

Those skilled in the art will recognize that a wide variety of position sensors and product detection sensors may be used with dispensing cartridges 400 and top shelf 320 and bottom shelf 340 of the refrigerated storage and dispensing unit 100 described herein without departing from the spirit and scope of the instant invention.

As mentioned above, given the modular construction of refrigerated storage and dispensing unit 100 discussed above, instead of dispensing cartridges 400, an alternative storage assembly may be provided as may be selected by persons skilled in the art. As shown in the side, cross-sectional view of FIG. 19, such alternative storage assembly may, in certain configurations, comprise an open-front storage box 500 equipped with a planar radio frequency identification (“RFID”) antenna 550, which may receive and store RFID tagged product containers.

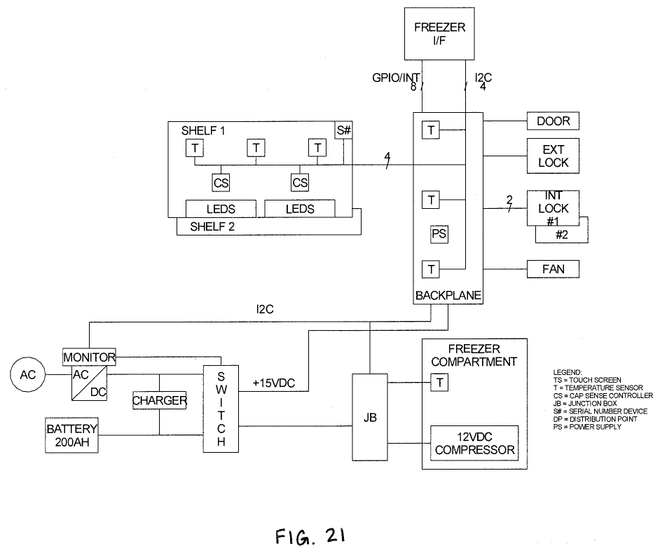

Open-front storage box 500 defines an open interior space 502 that may receive products of varying shapes and sizes, and thus may be used to store products that are not configured for storage in and dispensing from dispensing cartridges 400, but that are nonetheless desired to be stored with such products in a single refrigerated storage and dispensing system 100. With continuing reference to FIG. 19 and to the rear view of FIG. 20, open-front storage box 500 may have a bottom face 504, which bottom face 504 may be sized and otherwise configured to sit directly on flat panel 321 in the place of dispensing cartridges 400, or alternatively on bottom plate 326. Open-front storage box 500 may include ridges 514 on a back side thereof configured to engage with hooks 1214 of cartridge release mechanism 1210, thus allowing open-front storage box 500 to be locked to top shelf 320 or bottom shelf 340 in the same manner that dispensing cartridges 400 may be locked to top shelf 320 or bottom shelf 340. Open-front storage box 500 may include a slot 504 extending into the upper body of open-front storage box 500 from a back side thereof, which slot 504 receives planar RFID antenna 550. An electrical cable 552 may extend from planar antenna 550 out of slot 504 for connection to control compartment 115. Planar antenna 552 may be configured to read RFID tags positioned on products or product containers that are to be stored on open-front storage box 500, and more particularly to detect their placement on and removal from open-front storage box 500. Planar RFID antennas and RFID tags readable by them are readily commercially available and their construction is known to those skilled in the art, such that their specific configuration and operation is not further detailed here.

A junction box may control a heater for an ammonia absorption chiller or the compressor for maintaining a constant temperature in the chilled compartment of refrigerator cabinet 102. In certain configurations, the junction box may include a processor, SSR or SCR, I2C communication interface, NTC temperature probe, and power supply. If the chiller is ammonia absorption, the processor preferably runs a standard PID algorithm and numeral-impulse control with modifications for over-temperature. The over-temperature function is configured to shut down the heater when it appears that the chiller is overheating due to low gas, a bad or damaged heater, or high current. The nominal temperature setting for the inside of the chilled compartment will be controlled by the processor of control compartment 115 communicating with the junction box. Current status of the junction box may be read by the processor of the control compartment 115.

An AC/DC power supply with line filter is preferably used to provide a stable voltage for all of the electronics included in refrigerated storage and dispensing unit 100. The output of the power supply is preferably used to charge a backup battery. Both the output of the power supply and the backup battery are preferably controlled by a power switch. The power switch is preferably controlled by the processor of the control compartment 115 for selecting how the electronics and the chilled compartment 115 will be powered. The processor may monitor the main power supply and backup battery via the I2C communications interface.

In accordance with certain aspects of an embodiment of the invention, where a freezer compartment is included in refrigerated storage and dispensing unit 100, the freezer compartment preferably includes an enclosure with a compressor chiller, shelves for holding dispensing cartridges 400, an intermediate drawer assembly 360, dispensing sensing shelves, temperature sensing, LEDs, electric locks, a door sensor, a fan, a processor controlled thermostat, a power supply with UPS, and a communication link to chilled compartment or the control compartment 115. FIG. 21 provides a block diagram for the foregoing components. If provided, a freezer compartment preferably includes a freezer interface that contains I2C and GPIO signals necessary for communications with the processor of the control compartment 115. These signals are buffered and sent to the processor through the backplane 380. It is contemplated that a freezer compartment provided as herein will be configured in the same way as the chilled compartment and includes the same features, except that its temperature can be set below freezing to maintain products at lower temperatures than in the chilled compartment of refrigerator cabinet 102 discussed above.

All control and communications are preferably handled by the control compartment 115. The vending refrigerator may be configured to communicate with a server. In this configuration, the server consists of a computer running application software that is located at an off-site facility. This application software processes incoming data from the refrigerated storage and dispensing unit 100 and generates the data necessary for inventory control, maintenance, alarms, billing, and any other essential tasks. The data is also used to dispatch personnel for maintenance, customer support for alarms, and refilling.

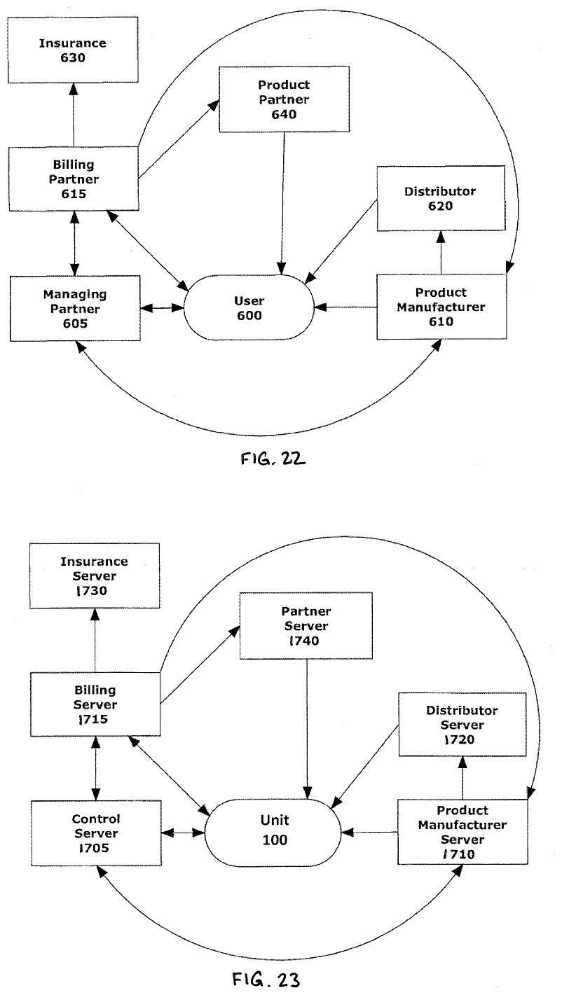

The server and refrigeration unit may communicate with one another, and form part of a fully integrated product supply, storage, and distribution system, as further described below, and as shown schematically in the exemplary flow diagram shown in FIG. 22.

The first element of the system is a user facility 600. The user facility 600 is the facility at which the refrigerated storage and dispensing unit 100 is placed. User facility 600 may be a physician's office, but it could be any other place at which the refrigerated storage and dispensing unit 100 is to be used, e.g., a pharmacy, a health department office, etc. Another element of the system is a product manufacturer 610, such as a company that manufactures the products that are to be dispensed through the vending refrigerator. An optional element of the system is a distributor 620, which is used by the manufacturer 610 to deliver the product to the user 600. A product partner 640 may also be provided that has the responsibility of managing the inventory of product in the refrigerated storage and dispensing unit 100. A managing partner 605 may be used in certain instances to control the functions of the refrigerated storage and dispensing unit 100. In some instances, the managing partner 605 is the manufacturer of the refrigerated storage and dispensing unit 100 and has responsibility for maintenance of the equipment and the data collected from the refrigerated storage and dispensing unit 100. A billing partner 615 may be responsible for invoicing and settling accounts with the various other members of the system. Insurance provider 630 may be another component, which is in communication with the billing partner 615 to manage payment for spent product.

The system is implemented through an integrated network as shown in FIG. 23. In accordance with certain aspects of an embodiment of the invention, the refrigerated storage and dispensing unit 100 is communicatively connected with the control server 1705 at the managing partner 605, the product manufacturer server 1710, the distributor server 1720, the partner server 1740, the billing server 1715, and the insurance server 1730. A person of ordinary skill in the art will recognize that the vending refrigerator need not be connected to all of the components listed above. For example, the refrigerated storage and dispensing unit 100 may be connected to the control server 1705 and the control server 1705 may be used to communicate with all other components as necessary.

The system described above is used to implement a method for distribution and dispensing of products. A product is first delivered to the user 600 from the manufacturer 610 or distributor 620. Data is collected from the control compartment 115 corresponding to the contents and operation of the refrigerated storage and dispensing unit 100 and sent to the control server 1705. The control server 1705 handles all transactions concerning the products dispensed through the refrigerated storage and dispensing unit 100. All data including alarms from the refrigerated storage and dispensing unit 100 are sent and processed by the control server 1705. The control server 1705 processes the data and determines whether the refrigerated storage and dispensing unit 100 needs to be refilled or if maintenance is required.

Optionally, a billing server 1715 at the billing partner 615 may receive information directly from the refrigerated storage and dispensing unit 100 or the control server 1705. The billing server 1715 manages billing to and from user 600, billing to and from insurance company 630, and orders stock from the product manufacturer 610. The billing server 1715 may also schedule visits from a product partner 640 to go to the user 600, while coordinating with the delivery of the product. The product partner 640 may be an organization with individuals qualified to store and maintain the physician's vaccine stock. In such instances, the product partner 640 preferably uses a CDC vaccine handling document in establishment of procedures. When a patient receives a vaccine, or other medication dispensed through the refrigerated storage and dispensing unit 100, the information is sent from a scanned vial or extracted from the POMIS (Physician's Office Management and Medical Information System) to the billing server 1715 to bill the insurance company 630 for the patient to whom the product was provided.

The control server 1705 will send stock refill requests to the billing server 1715 when stock levels fall below pre-determined amounts. The billing server 1715 needs to place such orders in a timely manner to ensure that the physician's office does not run out of any medication, e.g., a vaccine. The control server 1705 will track all on-hand inventory. The billing server 1715 will track all orders for deliveries and provide delivery information to the control server 1705. All alarms from the refrigerated storage and dispensing unit 100 in the physician's office will be sent via control server 1705 to the billing server 1715 for notification. Insurance 630 refers to entities that reimburse the product partner 640 for the cost of the drug, e.g., a vaccine, and reimburse the physician for administering the drug. Billing server 1715 will bill the insurance company 630 and dealing with managing the insurance payment transaction.

Manufacturer 610 refers to entities that are going to supply product, e.g., medications, vaccines, etc. to the user 600 or product partner 640. The manufacturer 610 receives at product manufacturer server 1710 an order from the billing server 1715. The order is then shipped directly to the user 600 or product partner 640 with the tracking number and manifest sent to the billing server 1715, which will relay that information to the control server 1705. It is contemplated that the product will arrive in a timely manner, e.g., within 48 hours of shipment.

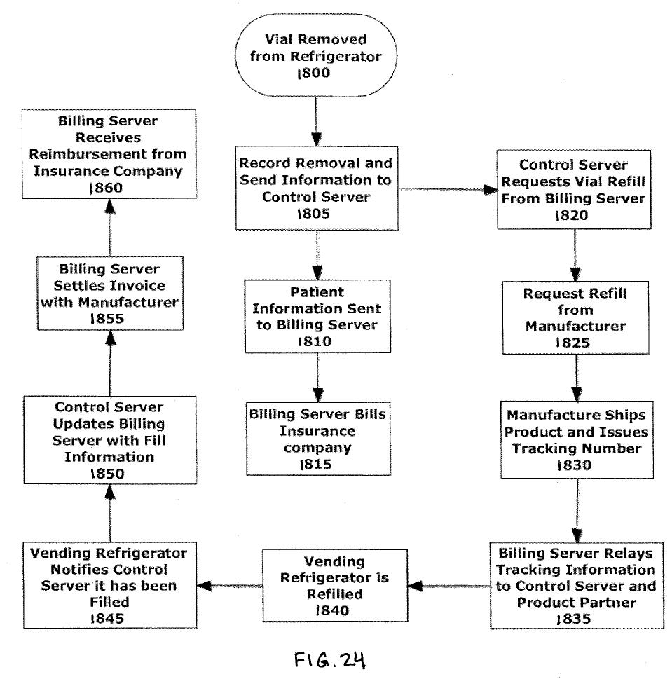

One exemplary method in accordance with an embodiment is provided in FIG. 24. In a first step 1800, an operator, such as a nurse in the physician office 600, removes a single product container (e.g., a vial stored in a dispensing cartridge 400) from the refrigerated storage and dispensing unit 100. In a second step 1805, the refrigerated storage and dispensing unit 100 records the removal and sends the information to control server 1705. In a third step 1810, the operator enters the information in the customer (e.g., patient) record and the data is sent to the billing server 1715 for billing by extracting the relevant information from the POMIS. In the next step 1815, the billing server bills the insurance company for reimbursement. At step 1820, the control server 1705 recognizes and tracks the use of the vial and, if the number of vials goes below a par level, communicates with the billing server 1715 to order more product. At step 1825, billing server 1715 requests a refill from manufacturer 610 by placing an order with the manufacturer 610 for more product.

The manufacturer 610 then ships product; generates a tracking number, manifest, and invoice; and sends the information to the billing server 1715 at step 1830. The billing server 1715 relays the tracking number and manifest to the control server 1705 and to the product partner 640 at step 1835. The refrigerated storage and dispensing unit 100 is then refilled at step 1840, and notifies the control server 1705 when it has been refilled at step 1845. Then, at step 1850, the control server 1705 informs billing server 1715 of product delivery and updated inventory levels. The billing server 115 then settles the manufacturer's invoice for the cost of the product at step 1855. The process is completed when the billing server 1715 receives reimbursement from insurance at step 1860.

An exemplary general system flow for the above-described system is as follows. It is contemplated that fill orders can be submitted through the keypad of refrigerated storage and dispensing unit 100, a smart phone, or a mobile device with a software application that receives information from the refrigerated storage and dispensing unit 100 and that can be communicatively connected with the system, e.g., the control server 1705 or billing server 1715. The filling system may also be controlled through a web page communicatively connected with the refrigerated storage and dispensing unit 100, e.g., through the control server 1705 or the billing server 1715.

The refrigerated storage and dispensing unit 100 may capture product information through the use of a bar code scanner. The bar code scanner may be located on the control compartment 115. The person that is charged with filling the refrigerated storage and dispensing unit 100 can scan each product, e.g., a vaccine, before re-filling dispensing cartridges 400. The refrigerated storage and dispensing unit 100 may contain a server within control compartment 115 that maintains communication between the user's office and a control server 1705 at a vendor that manufactures and maintains the refrigerated storage and dispensing unit 100. The refrigerated storage and dispensing unit 100 may report every movement of product and event or alarm requiring action to the manufacturer of refrigerated storage and dispensing unit 100.

As explained above, the dispensing cartridges 400 do not allow a product to be returned through the dispenser drawer 420. If a product is removed, it can be placed in drawer assembly 360. Optionally, products that allow for multiple dosages can be placed in drawer assembly 360 after initial dosing from such product. Preferably, refrigerated storage and dispensing unit 100 only tracks whether a product is in a dispensing cartridge 400. The user is responsible for tracking payments and distribution of the product in such multiple dosage cases.

A doctor and/or nurse may utilize the following exemplary process in using the refrigerated storage and dispensing unit 100. The doctor, nurse, or other user logs-in using his or her own unique user code. The user opens the door of refrigerated storage and dispensing unit 100 and pulls the required vial from the dispensing drawer 420 of the dispensing cartridge 400. If the user pulls the wrong vial, the vial may be placed in drawer assembly 360. Similarly, if the product is a multi-dose vaccine, the remainder may be placed in drawer assembly 360. The user then closes the door 105 of refrigerated storage and dispensing unit 100, provides the product to the patient or ultimate recipient, records the dosing in POMIS, and disposes of the vial or syringe under normal operation procedures. Dispensing is registered in refrigerated storage and dispensing unit 100 and relayed, for example, to the manufacturer of the refrigerated storage and dispensing unit 100 or any other party monitoring the refrigerator's use. The billing system is preferably configured to ensure the product, e.g., vaccine, is billed within 24 hours of use to the insurance company. Refill orders are preferably placed when stock levels are reduced or at specific intervals.

In a further exemplary process employed by a product specialist, the refrigerated storage and dispensing unit 100 may be optimally used in accordance with the following process. The manufacturer or manager of refrigerated storage and dispensing unit 100 establishes an initial inventory of product, e.g., vaccines. When a product vial is removed from the refrigerated storage and dispensing unit 100, the reduction of inventory is recorded in the control compartment 115 and relayed to the managing partner 605 or control server 1705. The managing partner 605 or control server 1705 recognizes when a stock level, and more preferably an unexpired stock level, falls below par and notifies the billing server 1715 or partner designated to place and manage product orders. The billing server 1715, or the partner designated to manage orders, places an order with the product manufacturer 610. The product manufacturer 610 ships the order to and alerts the billing server 1715 of the tracking information, which is then relayed to the control server or manufacturer/manager of the refrigerated storage and dispensing unit 100.

The billing server 1715 or party designated to manage orders notifies a product partner and the manufacturer/manager of the data received from the refrigerated storage and dispensing unit 100, including preferably that there are X deliveries of specific products occurring the following day for scheduling purposes. The product partner schedules a time to visit the user's office and replenishes the refrigerated storage and dispensing unit 100 from the delivered product order. If the delivery arrives prior to the arrival of the product partner specialist, the user may place the product in an appropriate refrigerator to maintain temperature control.

The product partner specialist verifies the thermal indicator on the box is in proper condition; logs in via the keypad using a unique code; unlocks the vaccine compartment; replaces the product in accordance with industry standards (e.g., using First-In, First-Out (FIFO) principle); and scans the bar code of each vial and interacts with the refrigerated storage and dispensing unit 100 front panel LCD/touchscreen to identify the product to be loaded. The refrigerated storage and dispensing unit 100 and the manager of the refrigerated storage and dispensing unit 100 or manufacturer's software preferably records the product name, the number of doses received, the date the product was received, the condition of the product on arrival, the product manufacturer, the lot number and product expiration date, as determined by the scanner and the front panel dialog. The refrigerated storage and dispensing unit 100 may include a flashing LED(s) under the cartridge/slot to indicate which one to fill, and the refiller preferably verifies that the vaccine being replaced is going into the correct cartridge/slot one at a time. Expired products are preferably removed and sent to the manufacturer for reimbursement and/or replacement. Expired vaccines may occur due to power outages, being past their expiration date, or temperature fluctuations.

It is contemplated that the control server 1705 may be configured to allow for the return of expired products. This option could be included under an inventory menu option entitled “return to manufacturer/expired product.” Expired products in the dispensing cartridges or in drawer assembly 360 can also be returned to the product manufacturer for disposal and/or reimbursement.

The product partner specialist ensures that all unexpired (and unrecalled) vials in drawer assembly 360 will remain there for future use. He or she then optionally locks the vaccine compartment, replaces any miscellaneous items as needed (labels, boxes, containers, etc.), and checks in with the user to see if there are any issues of any sort.

The product specialist also asks whether there are any maintenance items needing to be checked, and informs the manufacturer and/or manager of the refrigerated storage and dispensing unit 100. The temperature of the refrigerated storage and dispensing unit 100 is automatically controlled, such that there would be no need for the product specialist to record such information. The product specialist then provides a summary of product usage, preferably for the month, and answers any product related questions.

In accordance with an aspect of an embodiment of the invention, the system employs an exemplary inventory reconciliation process as follows. The product specialist may conduct a physical inventory of the refrigerated storage and dispensing unit 100 for reconciliation with the automated refrigerated storage and dispensing unit 100 and manufacturer/control server 1705 recorded inventory levels. The product specialist or other user conducting the inventory interacts with the refrigerated storage and dispensing unit 100 LCD/touchscreen 130 dialog or user input device 140 to note any and all inventory discrepancies. The recorded refrigerated storage and dispensing unit 100 and manufacturer/control server 1705 inventory levels are adjusted to match the physical inventory, with a time stamped note explaining the adjustment.

In some instances, it may become desirable to modify the input menus. An exemplary menu change process may include the following steps. If product demand is expected to change, e.g., required for back-to-school or flu season when certain vaccines will be in higher demand, then the menu and inventory may be changed. The product partner may evaluate the need for a second refrigerated storage and dispensing unit 100 or to change the quantity of product supplied. The product partner may submit a “menu change” to the manufacturer or manager of refrigerated storage and dispensing unit 100. The manager or manufacturer will then build the new menu and download the new menu to the refrigerated storage and dispensing unit 100 through the network. The manufacturer or manager of refrigerated storage and dispensing unit 100 may store all historical data on a server to be used for analysis as needed, such as location, product type (e.g., vaccine), and user. The product specialist arrives to the user's office and contacts the manager or manufacturer technical department for assistance with completing the menu change. The system is designed to include procedures when there is product remaining in the affected cartridge(s)/slot(s). For example, an “Extraction” option allows for movement of product without generating a billing invoice or replenishment order. The inventory is reloaded and the system is updated.

If there is a malfunction or an alarm is otherwise activated, the system is configured to employ the following process. Once an alarm is activated, the refrigerated storage and dispensing unit 100 records the alarm and relays notification of the alarm to the manufacturer/manager of the refrigerated storage and dispensing unit 100. The service telephone number is preferably displayed on the display of the refrigerated storage and dispensing unit 100. The manufacturer/manager may notify the product specialist as soon as possible via e-mail, SMS, or any other electronic means. The product specialist preferably contacts the user to determine the cause of the alarm (e.g., unplugged unit, temperature drop, etc.) and to resolve the problem (if able). The product specialist may follow a specified protocol. The result of the call is logged on the manufacturer/manager database for evaluation by management. The information collected may include the date, name of the person spoken to, time, and any other pieces of information deemed to be crucial by the user, manufacturer or service provider. The product specialist may contact the manufacturer/manager of the refrigerated storage and dispensing unit 100 for technical support if the issue remains unresolved. The manufacturer/manager may then send a technician as needed. Items that the technician may check include the length of back-up power available from the battery—48 hour minimum, 72 hour preferred, which allows for holiday weekends when the office may be closed for three days.

The following events are examples of items that might cause an alarm to be triggered: out of temperature range, door open beyond a predetermined period of time, no main power (i.e., switched over to battery), impending total power loss (i.e., no main and battery drained), no communication to server, illegal access or access try count exceeded, specific cartridge/slot not communicating, low batteries (test AC/DC load), dispensing an expired vaccine, and dispensing a recalled or quarantined vaccine. Records may be stored, such as for three years on the manufacturer's server, or as required by applicable law or business practice.

In an embodiment, the refrigerated storage and dispensing unit 100 may provide redundant systems to address possible power outages. The refrigerated storage and dispensing unit 100 may include a back-up battery to provide power in the event of a power failure. The refrigerated storage and dispensing unit 100 may be programed to lock and to not dispense product in order to maintain temperature and protect the stored products. A lock override upon entry of a special access code may be provided to allow emergency entry or for removal of product to another location. In addition, after a configurable time interval, the chilled or freezer compartments may be shut down to conserve battery power and the lock will continue to remain locked during this stage. Once the battery fails completely, the refrigerated storage and dispensing unit 100 will be left in an unlocked state.

A process may also be implemented to address recall and quarantine of stored products. In such event, the manufacturer/manager will record the recall/quarantine for a specific product, e.g., vaccine, including lot number on the refrigerated storage and dispensing unit 100. The product will be identified to be “On Hold” on the display of refrigerated storage and dispensing unit 100. In some embodiments, the dispensing cartridge 400 will be locked to prevent dispensation of the product. The refrigerated storage and dispensing unit 100 may also flash LED(s) to warn against use of a product, display a warning on the front panel display, raise an alarm if quarantined product is dispensed, and lock down under software-configurable conditions. A product specialist may remove recalled vaccine from refrigerated storage and dispensing unit 100 after entering a code and place such recalled vaccine in the user's refrigerator until resolved or remove it to its own facility.

The system can be further configured to allow transfer of vaccines between different locations, e.g., between physician offices in a vaccine partner territory. Software to implement the system may implement the following process. First, the manufacturer sets inventory levels for management of the process. The refrigerated storage and dispensing unit 100 and the manufacturer software has the ability to accept transfer vaccine from another user. The software may include transport practices established for a vaccine specialist, e.g., ability of a transport receptacle with 12-volt DC power convertor to cool the container as an option that can be plugged into electrical receptacles in a vehicle. The software also tracks temperature for cold chain. In some instances, the transport receptacle may be another refrigerated storage and dispensing unit 100 into which a dispensing cartridge 400 can be plugged, and that is powered by a car. This special software would be used for transport only.

The software and system may also include solutions for discrepancies and errors. In one exemplary embodiment, when shipping errors occur, the product specialist matches the billing server or partner shipping request to the product manufacturer shipping document and alerts that billing server or partner of discrepancies. The software may include a validation process to cross check that what was ordered is what was delivered and subsequently placed in the individual cartridge/slot at the dispensing cartridge 400.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It should be understood, therefore, that the invention may be practiced otherwise than as specifically set forth herein.

What is claimed is:

1. A refrigerated storage and dispensing unit, comprising:
a refrigerator cabinet;
a chassis positioned on an interior of said refrigerator cabinet having walls defining an open cavity on an interior of said chassis;
a shelf affixed to said walls of said chassis within said open cavity; and
a plurality of product dispensing cartridges removably positioned on said shelf, each said product dispensing cartridge further comprising a vertical housing holding a plurality of product units and a dispensing drawer configured for horizontal movement out of said housing, wherein each said product dispensing cartridge is configured to sequentially dispense a single one of said product units upon each horizontal extension of said dispensing drawer from a fully closed position to a fully open position.
